(19) United States
(12) Patent Application Publication
(10) Pub. No.: US 2014/0255999 A1
(43) Pub. Date: Sep. 11, 2014

(54) SIMPLE SUGAR STARVED LIGNOCELLULOSIC BIOMASS ENZYME PRODUCTION

(71) Applicant: C5-6 Italy S.R.L., Tortona (IT)

(72) Inventors: Laura Volpati, Milano (IT); Beatriz Rivas Torres, Arenzano (IT); Stefano Paravisi, Urganano (IT)

(21) Appl. No.: 14/350,922

(22) PCT Filed: Oct. 12, 2012
(86) PCT No.: PCT/EP2012/070340

§ 371 Date: Apr. 10, 2014

(30) Foreign Application Priority Data
Oct. 13, 2011 (IT) TO2011A000918

(51) Int. Cl. C12P 19/14; C12P 19/02

(52) U.S. Cl. CPC C12P 19/14; C12P 19/02
USPC 435/99

(57) ABSTRACT

This specification discloses a process for producing at least one enzyme from a host cell for the hydrolysis of a first pre-treated ligno-cellulosic biomass under simple sugar starved conditions wherein the cultivation environment has very little, preferable no, simple sugars added other than those present in a ligno-cellulosic biomass used to feed and grow the host cell. The cultivation environment is substantially void of fermentation stimulators and inducers of enzyme production. Preferably, the cultivation environment has a high dry matter content of the pre-treated ligno-cellulosic biomass.

### BACKGROUND

It is known that the hydrolysis of cellulose and hemicellulose from ligno-cellulosic feedstocks requires a well-balanced mixture of enzymes consisting of endoglucanases, cellobiohydrolases, β-glucosidases, xylanases, mannanases and various enzymes acting on side chains of xylans and mannans. Enzyme production is an important step in the biomass-toethanol process as enzyme or enzyme mixture production and application are currently among the more costly processing steps for biologically based routes to ligno-cellulose material utilization.

The enzyme systems of the plant degrading fungus Trichoderma reesei are the most extensively investigated and believed to be the most widely organism used to obtain commercial enzyme mixtures. T. reesei produces numerous cellulose- and hemicellulose-degrading enzymes even if extracellular β-glucosidase secretion is low. As it is well known that β-glucosidase activity content is critical in order to obtain high cellulose conversion, T. reesei enzyme solution is commonly supplemented with β-glucosidases to obtain a well-balanced enzyme solution and further advance the hydrolysis of the cellulose. In other cases commercial enzyme or enzyme mixture solutions could also be obtained using enzymes produced by other, good β-glucosidase-producing fungi.

Even if no clear relationship between the substrate used for cultivation and the hydrolytic performance of the resulting enzymes on the particular substrates has been reported in previous studies on T. reesei Rut C30 (See Juhasz, T., Szengyel, Z., Reczey, K., Siika-Aho, M., Viikari, L. (2 Characterization of enzyme or enzyme mixtures and hemienzyme or enzyme mixtures produced by Trichoderma reesei on various carbon sources. Process Biochem, 40, 3519-3525) or Penicillium brasilianum, (See Jorgensen, H., Olsson, L. (2 Production of enzyme or enzyme mixtures by Penicillium brasilianum IBT 20888: Effect of substrate on hydrolytic performance. Enzyme Microb Technol, 38, 381-390) many research groups had tried to produce enzyme or enzyme mixtures using in some manner the same material used for bio-ethanol production to reduce final enzyme mixture costs.

One such process is described in WO 2007005918. This process adds the described pre-treated ligno-cellulosic substrate as an inducer of enzyme growth, while using constant addition of glucose as the feed for the organism growth. The stated purpose of this technology is to replace pure cellulose used today for enzyme or enzyme mixture production in a host cell, with pre-treated ligno-cellulosic material, such as, especially pre-treated Arundo Donax (PCS) as described in WO 2007005918. The advantage of this is that the production cost is reduced due to use of an inducer (ligno-cellulosic biomass) which is easily available and thus cheaper than pure cellulose. As an inducer, WO 2007005918 uses a small amount of biomass and continually adds glucose to feed the organism.

Even by using ligno-cellulosic biomass as an inducer, the cost of enzymes remains high and it is key factor limiting the diffusion of the enzymatic technologies. The main factors limiting the cost decrease of enzymes are represented by the cost of the ingredients used for promoting the growth of the micro-organism and operating costs. Among the ingredients, main costs are represented by the glucose, fermentation stimulators such as vitamins and mineral salts, and inducers of enzyme production, such as lactose and Isopropyl 13-D-1-thiogalactopyranoside (IPTG). Fermentation stimulators and inducers of enzyme production are expensive even if used in small amount. On the side of operating costs, the energy needed for agitating the fermentation medium and the total fermentation time are to be considered.

There is therefore the need of an inexpensive process for producing an enzyme or enzyme mixture.

### SUMMARY

Disclosed in this specification is a process for producing at least one enzyme from a host cell for the hydrolysis of a first pre-treated ligno-cellulosic biomass wherein said process comprises the steps of cultivating the host cell which is capable of producing the at least one enzyme for a cultivation time, wherein the cultivation of the host cell occurs in a cultivation environment comprising the host cell, a second pre-treated ligno-cellulosic biomass, said second pre-treated ligno-cellulosic biomass comprised of complex sugars, and optionally simple sugars; the cultivation is done under simple sugar starved conditions of having an optional added simple sugar in the range of 0 to 10% by weight of the cultivation environment on a dry basis for at least a portion of the cultivation time; and the cultivation environment is substantially void of added vitamins, and/or added minerals and/or added inducers of enzymes production.

It is also disclosed that the dry matter content by weight of the second pre-treated ligno-cellulosic biomass in the cultination environment may be higher than 2%, preferably higher than 4%, more preferably higher than 6%, even more preferably higher than 8%, yet even more preferably higher than 10%, most preferably higher than 15%.

It is further disclosed that the portion of the cultivation time under simple sugar starved conditions may be selected from the group consisting of at least 50% of the cultivation time, at least 75% of the cultivation time, at least 85% of the cultivation time, at least 90% of the cultivation time, at least 98% of the cultivation time, and a period equal to the cultivation time.

It is further disclosed that the optional added simple sugar may be in a range selected from the group consisting of 0 to 5% by weight of the cultivation environment on a dry basis, 0 to 2.5% by weight of the cultivation environment on a dry basis, 0 to 2.0% by weight of the cultivation environment on a dry basis, 0 to 1.0% by weight of the cultivation environment on a dry basis and no optional simple sugar.

It is further disclosed that the enzyme or enzyme mixture may be harvested by removing the enzyme or enzyme mixture from the cultivation environment and that it may be further used to hydrolyze the first ligno-cellulosic biomass and the first ligno-cellulosic biomass and the second pretreated ligno-cellulosic biomass both comprise ligno-cellulosic biomass derived from group consisting of the same grass genus or more preferably the same grass species.

It is also disclosed that there is an enzyme produced, a hydrolyzed ligno-cellulosic biomass by the enzyme or enzyme mixture produced under the simple sugar starved conditions.

### DETAILED DESCRIPTION

It has been discovered a process for producing an enzyme or enzyme mixture from a host cell by cultivating the host cell on a minimal cultivation environment, thereby proving a method for producing enzymes at a low cost.

According to one aspect of the invention, it has been discovered that the activity on a pre-treated ligno-cellulosic biomass material of an enzyme or enzyme mixture produced from a host cell can be enhanced when the simple sugars such as glucose and xylose used to traditionally feed host cells are replaced with the pre-treated ligno-cellulosic material, such as, pre-treated Arundo Donax. The pre-treated ligno-cellulosic material is preferably pre-treated by soaking and washing in hot water and pressing to remove the water and water soluble compounds. The soaking pre-treatment removes the soluble sugar monomers (xylose and glucose). Besides of enhancing the activity of the enzyme produced, the pre-treated ligno-cellulosic material is significantly less expensive than glucose and xylose sources used for traditionally feeding the host cells. Moreover, a significant reduction of time is obtained, when the pre-treated ligno-cellulosic material is used as the unique carbon source, and no or few added simple sugars are added to the cultivation environment.

According to another aspect of the invention, it has been discovered that the cultivation of the host cell producing the enzyme or enzyme mixture does not require the addition of expensive fermentation stimulators, such as vitamins and minerals or mineral salts, and/or added enzyme producing inducers, such as lactose and ITPG, thereby the cultivation environment comprising the pre-treated ligno-cellulosic material is void, or substantially void, of added fermentation stimulators and added inducers of enzyme production. In the present disclosure, by the expression “substantially void of added fermentation stimulators” it is meant that the concentration in the cultivation environment of each added fermentation stimulator (both vitamin and minerals) is less than 1 g/l, more preferably less than 500 mg/l, even more preferably less than 200 mg/l, yet even more preferably less than 100 mg/l, yet even more preferably less than 50 mg/l, yet even more preferably less than 10 mg/l, yet even more preferably less than 5 mg/l, most preferably less than 2 mg/l. These concentration values are significant less than the concentration values usually used in similar processes known in the art. In the present disclosure, by the expression “substantially void of added inducers of enzyme production” it is meant that the concentration in the cultivation environment of each added inducers of enzyme production (both lactose and ITPG) is less than 100 mg/l, more preferably less than 50 mg/l, even more preferably less than 20 mg/l, yet even more preferably less than 10 mg/l, most preferably less than 5 mg/l. These concentration values are significant less than the concentration values usually used in similar processes known in the art.

According to a further aspect of the invention, it has been discovered that the cultivation of the host cell producing the enzyme or enzyme mixture may occur in a cultivation environment having a dry matter content by weight of the pretreated ligno-cellulosic material substantially higher than previously disclosed method. The dry matter content by weight of the pre-treated ligno-cellulosic material in the cultivation environment may higher than 2%, preferably higher than 4%, more preferably higher than 6%, even more preferably higher than 8%, yet even more preferably higher than 10%, most preferably higher than 15%. The increase in the dry matter content reduces the energy per gram of the cultivation environment required for agitating the cultivation environment, with respect to previously disclosed processes. Moreover, the amount of cultivation environment needed for producing a certain amount of enzymes is reduced, and correspondingly the volume of the equipment (bio-reactor), thereby generating a further reduction in investment costs.

A further advantage of the invention is that the enzyme mixture coming from the host cell is more reactive to the pretreated ligno-cellulosic material used to feed and grow the host cell when the enzyme mixture is used to hydrolyze similar pre-treated ligno-cellulosic materials.

A person skilled in the art will appreciate that the features of the disclosed process significantly reduce the production cost of enzymes.

### Process of Producing Enzymes

It is well known in the art to produce enzyme or enzyme mixture in a host cell of fungal origin, such as filamentous fungi, or bacteria origin. The growth process of the invention may be a well known process, except that the feed, such as pure glucose, is limited and the primary feed be pretreated ligno-cellulosic material.

Enzyme production procedures are well known in the art. In context of the present invention the enzyme or enzyme mixture is preferably an extra-cellular enzyme or enzyme mixture secreted into the fermentation medium by the host cell. Alternatively, the enzyme or enzyme mixture is intracellular.

A host cell capable of producing enzyme or enzyme mixture is grown under precise cultural conditions at a particular growth rate. When the host cell culture is introduced into the fermentation medium the inoculated culture passes through a number of stages. Initially growth does not occur. This period is referred to as the lag phase and may be considered a period of adaptation. During the next phase referred to as the “exponential phase” the growth rate of the host cell culture gradually increases. After a period of maximum growth the rate ceases and the culture enters stationary phase. After a further period of time the culture enters the death phase and the number of viable cells declines. When, in the growth phase the enzyme, or enzyme mixture of interest is expressed depends on the enzyme of interest and the host cell. The enzyme or enzyme mixture may, in one embodiment, be expressed in the exponential phase. In another embodiment, the enzyme or enzyme mixture may be produced in the transient phase between the exponential phase and the stationary phase. The enzyme or enzyme mixture may also, in another embodiment, be expressed in the stationary phase and/or just before sporulation. The enzyme or enzyme mixture may, according to the invention, also be produced in more than one of the above mentioned phases.

In other words, according to the invention the host cell is cultivated in a suitable medium and under conditions allowing at least one enzyme or an enzyme mixture to be expressed, preferably secreted and optionally recovered. While as noted above, the host cell growth has many technical phases, for the purposes of this specification, these phases are grouped together in the term cultivation. Host cell cultivation takes place in a fermentation medium comprising a carbon source and a pre-treated ligno-cellulosic material as feed. According to a preferred embodiment the pre-treated ligno-cellulosic material has been pre-treated by being soaked/washed and then steam exploded as described in WO 2010113129, the teachings of which are incorporated by reference.

After fermentation the enzyme or enzyme mixture may optionally be recovered using methods well known in the art. For example, extra-cellular enzyme or enzyme mixture recovery from the fermentation medium may be done using conventional procedures including, but not limited to, centrifugation, filtration, extraction, spray-drying, evaporation, or precipitation. Procedures for recovery of an intracellular enzyme or enzyme mixture are also well known in the art.

At least in context of the present invention the interchangeable terms “cultivation” and “fermentation” means any process of producing an enzyme or enzyme mixture using a mass culture consisting of one or more host cells. The present invention is useful for especially industrial scale production, e.g., having a culture medium of at least 50 litres, preferably at least 1 litres, more preferably at least 5 litres.

The enzyme or enzyme mixture may include, but is not limited to any of those belonging to the group of enzyme or enzyme mixture comprising endoglucanases (endo-1,4-β-D-glucanase), cellobiohydrolases or exoglucanases (exo-1,4-β-D-glucanase), β-glucosidase (1,4-β-D-glucosidase), endo-1,4-β-xylanase, endo-1,4-β-mannanase, 1,4-β-xylosidase, 1,4-β-mannosidase.

A process of the invention may be performed as a batch, a fed-batch, a repeated fed-batch or a continuous process.

A process of the invention may be carried out aerobically or anaerobically. Some enzymes are produced by submerged cultivation and some by surface cultivation. Submerged cultivation is preferred according to the invention.

Thus, according to one aspect, the invention relates to processes of producing an enzyme or enzyme mixture in a host cell comprising cultivating said host cell capable of producing enzyme or enzyme mixture under conditions conducive for production of an enzyme or enzyme mixture, such as enzyme or enzyme mixture, wherein pre-treated ligno-cellulosic material is used to grow the host cell under simple sugar starved conditions.

More specifically, the described process produces at least one enzyme from a host cell for the hydrolysis of a first pre-treated ligno-cellulosic biomass wherein said process comprises the steps of first cultivating the host cell which is capable of producing the at least one enzyme for a cultivation time, wherein the cultivation of the host cell occurs in a cultivation environment comprising the host cell, a second pre-treated ligno-cellulosic biomass, said second pretreated ligno-cellulosic biomass comprised of complex sugars, and optionally simple sugars; and the cultivation is done for at least a portion of the cultivation time under simple sugar starved conditions wherein the cultivation environment may further comprise an optional added simple sugar in the range of 0 to 10% by weight of the cultivation environment on a dry basis.

The cultivation time is the amount of time measured from the addition of the pre-culture volume to host cell cultivation environment to the harvest, removal, or separation of the enzyme or enzyme mixture from the cultivation environment. In the case of multiple removals, the cultivation time ends at the time when the first enzyme or enzyme mixture is harvested from the culture medium.

Complex sugars are those sugars which are not monomeric sugars. Simple sugars are the monomeric sugars, and may selected from the group consisting of glucose, xylose, arabinose, mannose, galactose, and fructose. It should be noted that there may be other simple sugars not in the preceding list.

The amount of ligno-cellulosic biomass present in the cultination environment should be sufficient for the growth of the host cell to produce the amount of the enzyme or enzyme mixture desired.

The phrase simple sugar starved conditions means generally that more than 50% by weight of the host cell feed is from the pre-treated ligno-cellulosic biomass and not from added simple sugars. An exemplary simple sugar starved condition is when the amount of optional simple sugars added to the process, if any is added at all, is in the range of 0 to 10% by weight of the cultivation environment on a dry basis. More preferably, the optional simple sugars added should be in the range of 0 to 5% by weight of the cultivation environment on a dry basis, with 0 to 2.5% by weight being even more preferred, with 0 to 2.0% being the most preferred (if simple sugars are added at all). In the best case, there are no simple sugars added which is the perfect simple sugar starved condition. Additionally, the phrase simple sugars added means that there could be one or more simple sugars added.

The presence of the optional simple sugars added can also be expressed as the ratio of the amount of the optional simple sugars added to the amount of sugars from the pre-treated ligno-cellulosic biomass. The most preferred ratio is 0.0, which is the absence of any optional simple sugars. However, the ratio should be preferably less than 2.0 or 1.5, with less than 1.12 being more preferred, less than 0.53 being even more preferred and less than 0.33 also being a preferred value. In one embodiment the optional simple sugar is present, but at less than the percentage indicated or present less than the ratio indicated.

The simple sugar starved conditions should be maintained for at least a portion of the cultivation time. Expressed quantitatively, the simple sugar starved conditions should be maintained for at least 50% of the cultivation time, with 75% being more preferred, 85% being even more preferred, with 95% being even yet more preferred with 99 and 100% of the cultivation time being the most preferred. 100% of the cultivation time is when the at least a portion of the cultivation time equals the cultivation time.

In this manner, the growth of the host cell and its enzymes are influenced by the pre-treated ligno-cellulosic biomass feed of the second ligno-cellulosic biomass and adapt themselves over time to better hydrolyze the pre-treated ligno-cellulosic biomass. In this manner, when the enzyme mixture is used to hydrolyze a ligno-cellulosic biomass, especially a pre-treated ligno-cellulosic biomass having a similar composition as the second pre-treated ligno-cellulosic biomass feed, there is more reactivity (i.e. better enzymatic hydrolysis) to the pre-treated ligno-cellulosic biomass.

### Substrate and Additives

The substrate used in a process of the invention may be any substrate known in the art. Suitable substrates are available from commercial suppliers or may be prepared according to published compositions (e.g., in catalogues of the American Type Culture Collection).

Carbon source substrates commonly used as feed for enzyme or enzyme mixture production includes glucose or similar sugars, provided their consumption relative to the consumption of the complex sugars is within the specified boundaries. Nitrogen source substrates such us ammonia (NH₄Cl) or urea, may be added to improve cultivation and enzyme or enzyme mixture production. A key feature of the disclosed process is that no fermentation stimulators, with the exception of those already comprised in the pre-treated ligno-cellulosic material, are added to the cultivation environment, or are added in a small amount, that is the cultivation environment does not comprise added fermentation stimulators or comprises added fermentation stimulators in smaller amount with respect to previously known processes. In the context of the present disclosure, added fermentation stimulators for growth include vitamins and minerals. Vitamins which are not added or added in small amount are selected from the group consisting of: biotin, pantothenate, nicotinic acid, meso-inositol, thiamine, pyridoxine, para-aminobenzoic acid, folic acid, riboflavin, and Vitamins A, B, C, D, and E. Minerals which are not added or added in a small amount are minerals and mineral salts that supply nutrients selected from the group consisting of: B, P, Mg, S, Ca, Fe, Zn, Mn, Co, Mo and Cu.

A key feature of the disclosed process is that no inducers of enzyme production, with the exception of those already comprised in the pre-treated ligno-cellulosic material, are added to the cultivation environment, or are added in a small amount, that is the cultivation environment does not comprise added inducers of enzyme production or comprises added inducers of enzyme production in smaller amount with respect to previously known processes. In the context of the present disclosure, inducers of enzyme production which are not added or added in a small amount to the cultivation environment are selected from the group consisting of lactose and ITPG.

Pure cellulose, usually used as an inducer (and carbon source) in enzyme or enzyme mixture production processes, is replaced with pre-treated ligno-cellulosic material, preferably detoxified if acid pre-treated, such as washed, pretreated ligno-cellulosic material.

The pre-treated ligno-cellulosic material is a carbon source and may be added to the culture medium together with a carbon source, but may also be added separate from the carbon source. According to the invention the pre-treated ligno-cellulosic material may be added to the culture medium either prior to innoculation, simultaneously with innoculation or after innoculation of the host cell culture in an amount at least corresponding to the amount of available complex sugars needed to grow the host cell. When ligno-cellulosic biomass is added during the cultivation time, a new calculation of the amount optional simple sugars added or the ratio of optional simple sugars to ligno-cellulosic biomass is done. While the amount of simple sugars may not have been low enough during the initial part of the cultivation time, by adding ligno-cellulosic biomass to the cultivation environment, the amount of optional simple sugars added would fall within the specified ranges, at least for the time remaining in the cultivation time.

A person skilled in the art can easily determine when to add and what amount of pre-treated ligno-cellulosic material to add during a enzyme or enzyme mixture producing process of the invention. During the time span of cultivation pretreated ligno-cellulosic material is preferably added in amounts corresponding to the activity of glucose normally consumed by the cell host and kept within the previously specified limits.

As mentioned above pre-treated ligno-cellulosic material is used the same way glucose is normally used in well known enzyme or enzyme mixture production processes.

For instance, when producing enzyme or enzyme mixture using a strain of Penicillium, such as Penicillium decumbens, as the host cell, the glucans and xylans as present in the pretreated ligno-cellulosic material were charged at a level of 2.7 total grams/L. A process of the invention may last for the same period of time as a corresponding traditional process, such as between 3 and 10 days. Penicillium fermentations, including Penicillium decumbens fermentations, in general last for between 3-9 days.

### Ligno-Cellulosic Material

According to the invention “ligno-cellulosic material” includes any material that comprises ligno-cellulose. Ligno-cellulose is generally found, for example, in the stems, leaves, hulls, husks, and cobs of plants or leaves, branches, and wood of trees. The ligno-cellulosic material can also be, but is not limited to, herbaceous material, agricultural residues, forestry residues, municipal solid wastes, waste paper, and pulp and paper mill residues. It is understood herein that ligno-cellulosic material may be in the form of plant cell wall material containing lignin, cellulose, and hemicellulose in a mixed matrix.

In an embodiment the ligno-cellulosic material is corn fiber, rice straw, pine wood, wood chips, poplar, wheat straw, switch grass, bagasse, Arundo donax, myscanthus, eucalyptus, bamboo, paper and pulp processing waste. In a preferred embodiment the ligno-cellulosic material is Arundo Donax. In another preferred embodiment the ligno-cellulosic material is woody or herbaceous plants selected from the group consisting of the grasses. Alternatively phrased, the preferred ligno-cellulosic biomass is selected from the group consisting of the plants belonging to the Poaceae or Gramineae family. The role of starch may be present but in naturally occurring amounts. The ligno-cellulosic biomass preferably has less than 70% by dry weight, with less than 50% starch by dry weight being more preferred and less than 25% by dry weight being most preferred.

### Pre-Treatment

According to the invention ligno-cellulosic material is pretreated. The term “pre-treated” may be replaced with the term “treated”. However, preferred techniques contemplated are those well known for “pre-treatment” of ligno-cellulosic material as will be describe further below.

As mentioned above treatment or pre-treatment may be carried out using conventional methods known in the art, which promotes the separation and/or release of cellulose and increased accessibility of the cellulose from ligno-cellulosic material.

Pre-treatment techniques are well known in the art and include physical, chemical, and biological pre-treatment, or any combination thereof. In preferred embodiments the pre-treatment of ligno-cellulosic material is carried out as a batch or continuous process.

Physical pre-treatment techniques include various types of milling/comminution (reduction of particle size), irradiation, steaming/steam explosion, and hydrothermolysis, in the preferred embodiment, soaking, removal of the solids from the liquid, steam exploding the solids to create the pre-treated ligno-cellulosic biomass.

Comminution includes dry, wet and vibratory ball milling. Preferably, physical pre-treatment involves use of high pressure and/or high temperature (steam explosion). In context of the invention high pressure includes pressure in the range from 3 to 6 MPa preferably 3.1 MPa. In context of the invention, high temperature include temperatures in the range from about 100 to 300° C., preferably from about 160 to 235° C. In a specific embodiment impregnation is carried out at a pressure of about 3.1 MPa and at a temperature of about 235° C. In a preferred embodiment the physical pre-treatment is done according to the process described in WO 2010/113129, the entire teachings of which are incorporated by reference.

Although not needed or preferred, chemical pre-treatment techniques include acid, dilute acid, base, organic solvent, lime, ammonia, sulfur dioxide, carbon dioxide, pH-controlled hydrothermolysis, wet oxidation, and solvent treatment.

If the chemical treatment process is an acid treatment process, it is more preferably, a continuous dilute or mild acid treatment, such as treatment with sulfuric acid, or another organic acid, such as acetic acid, citric acid, tartaric acid, succinic acid, or any mixture thereof. Other acids may also be used. Mild acid treatment means at least in the context of the invention that the treatment pH lies in the range from 1 to 5, preferably 1 to 3.

In a specific embodiment the acid concentration is in the range from 0.1 to 2.0 wt % acid, preferably sulfuric acid. The acid is mixed or contacted with the ligno-cellulosic material and the mixture is held at a temperature in the range of around 160-220° C. for a period ranging from minutes to seconds. Specifically the pre-treatment conditions may be the following: 165-183° C., 3-12 minutes, 0.5-1.4% (w/w) acid concentration, 15-25, preferably around 20% (w/w) total solids concentration. Other contemplated methods are described in U.S. Pat. Nos. 4,880,473, 5,366,558, 5,188,673, 5,705,369 and 6,228,177.

Wet oxidation techniques involve the use of oxidizing agents, such as sulfite based oxidizing agents and the like. Examples of solvent treatments include treatment with DMSO (Dimethyl Sulfoxide) and the like. Chemical treatment processes are generally carried out for about 5 to about 10 minutes, but may be carried out for shorter or longer periods of time.

Biological pre-treatment techniques include applying ligninsolubilizing micro-organisms (see, for example, Hsu, T.-A., 1996, Pre-treatment of biomass, in Handbook on Bioethanol:

Production and Utilization, Wyman, C. E., ed., Taylor & Francis, Washington, D.C., 179-212; Ghosh, P., and Singh, A., 1993, Physicochemical and biological treatments for enzymatis/microbial conversion of ligno-cellulosic biomass, Adv. Appl. Microbiol. 39: 295-333; McMillan, J. D., 1994, Pretreating ligno-cellulosic biomass: a review, in Enzymatic Conversion of Biomass for Fuels Production, Himmel, M. E., Baker, J. 0., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of ligno-cellulosic hydrolysates for ethanol production, Enz. Microb. Tech. 18: 312-331; and Vallander, L., and Eriksson, K.-E. L., 1990, Production of ethanol from ligno-cellulosic materials: State of the art, Adv. Biochem. Eng./Biotechnol. 42: 63-95).

In an embodiment both chemical and physical pre-treatment is carried out including, for example, both mild acid treatment and high temperature and pressure treatment. The chemical and physical treatment may be carried out sequentially or simultaneously.

In a preferred embodiment the pre-treatment is carried out as a soaking step with water at greater than 1° C., removing the ligno-cellulosic biomass from the water, followed by a steam explosion step.

In a preferred embodiment the pre-treated ligno-cellulosic material is comprised of complex sugars, also known as glucans and xylans (cellulose and hemicellulose) and lignin.

### Enzyme or Enzyme Mixture

A enzyme or enzyme mixture means a cellulolytic enzyme or mixture of enzymes capable of degrading ligno-cellulosic biomass. An enzyme or enzyme mixture produced according to the described process may be of any origin including of bacterial or fungal origin. Chemically modified or protein engineered variants are included. Suitable enzyme or enzyme mixtures include enzyme or enzyme mixtures from the general Cellulomonas, Bacillus, Pseudomonas, Humicola, Fusarium, Thielavia, Acremonium, Chrysosporium, Penicillium, Themobifida and Trichoderma, e.g., fungal enzyme or enzyme mixtures produced by Humicola insolens, Themobifida fusca, Cellulomonas fimi, Myceliophthora thermophila, Thielavia terrestris, Fusarium oxysporum, Chrysosporium lucknowense, Penicillium decumbens, and Trichoderma reesei.

In an embodiment the enzyme or enzyme mixture produced is an enzyme or enzyme mixture complex homologous to the host cell. In an embodiment the enzyme or enzyme mixture produced is a enzyme or enzyme mixture complex homologous to a host cell of the genus Penicillium, preferably a strain of Penicillium decumbens.

It is to be understood that the enzyme or enzyme mixture produced may also be a mono-component enzyme or enzyme mixture, e.g., comprise an endoglucanase, exo-cellobiohydrolase, glucohydrolase, or beta-glucosidase produced recombinantly in a suitable host cell. Suitable host cells are described further below.

The enzyme or enzyme mixture produced may also be a enzyme or enzyme mixture preparation where one or more homologous enzyme or enzyme mixture components are deleted or inactivated from the host cell natively producing the enzyme or enzyme mixture.

### Host Cell Capable of Producing an Enzyme or Enzyme Mixture

The host cell may be of any origin. As mentioned above the enzyme or enzyme mixture may be homologous or heterologous to the host cell capable of producing the enzyme or enzyme mixture.

The term “recombinant host cell”, as used herein, means a host cell which harbours gene(s) encoding enzyme or enzyme mixture and is capable of expressing said gene(s) to produce enzyme or enzyme mixture, wherein the enzyme or enzyme mixture coding gene(s) have been transformed, transfected, transducted, or the like, into the host cell. The transformation, transfection, transduction or the like technique used may be well known in the art. In a preferred embodiment the gene is integrated into the genome of the recombinant host cell in one or more copies.

When the enzyme or enzyme mixture is heterologous the recombinant host cell capable of producing the enzyme or enzyme mixture is preferably of fungal or bacterial origin. The choice of recombinant host cell will to a large extent depend upon the gene(s) coding for the enzyme or enzyme mixture and the origin of the enzyme or enzyme mixture.

The term “wild-type host cell”, as used herein, refers to a host cell that natively harbours gene(s) coding for enzyme or enzyme mixture and is capable of expressing said gene(s). When the enzyme or enzyme mixture is a homologous preparation or enzyme or enzyme mixture complex the wild-type host cell or mutant thereof capable of producing the enzyme or enzyme mixture is preferably of fungal or bacterial origin.

A “mutant thereof” may be a wild-type host cell in which one or more genes have been deleted or inactivated, e.g., in order to enrich the enzyme or enzyme mixture preparation in a certain component. A mutant host cell may also be a wild-type host cell transformed with one or more additional genes coding for additional enzymes or proteins in order to introduce one or more additional enzyme activities or other activities into the enzyme or enzyme mixture complex or preparation natively produced by the wild-type host cell. The additional enzyme(s) may have the same activity (e.g., enzyme or enzyme mixture activity) but merely be another enzyme molecule, e.g., with different properties. The mutant wild-type host cell may also have additional homologous enzyme coding genes transformed, transfected, transducted, or the like, preferably integrated into the genome, in order to increase expression of that gene to produce more enzyme.

In a preferred embodiment the recombinant or wild-type host cell is of filamentous fungus origin. Examples of host cells include the ones selected from the group comprising Acremonium, Aspergillus, Aureobasidium, Bjerkandera, Ceriporiopsis, Chrysosporium, Coprinus, Coriolus, Cryptococcus, Filobasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a more preferred embodiment the filamentous fungal host cell is selected from the group comprising a strain of Aspergillus awamori, Aspergillus fumigatus, Aspergillus foetidus, Aspergillus japonicus, Aspergillus nidulans, Aspergillus niger or Aspergillus oryzae. In an even more preferred embodiment, the strain is Penicillium decumbens.

In another preferred embodiment the filamentous fungal host cell is a strain of Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another preferred embodiment, the filamentous fungal host cell is selected from the group comprising a strain of Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, or Ceriporiopsis subvermispora, Chrysosporium lucknowense, Coprinus cinereus, Coriolus hirsutus, Humicola insolens, Humicola lanuginosa, Mucor miehei, Myceliophthora thermophila, Neurospora crassa, Penicillium purpurogenum, Penicillium decumbens, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride.

In another preferred embodiment the recombinant or wild-type host cell is of bacterial origin. Examples of host cells include the ones selected from the group comprising gram positine bacteria such as a strain of Bacillus, e.g., Bacillus alkalophilus, Bacillus amyloliquefaciens, Bacillus brevis, Bacillus circulans, Bacillus coagulans, Bacillus lautus, Bacillus lentus, Bacillus lichenifonnis, Bacillus megaterium, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp.

### Use

In another aspect the process relates to the use of pretreated ligno-cellulosic material as a carbons source feed for producing an enzyme or enzyme mixture in a host cell.

The process also relates to the use of pre-treated ligno-cellulosic material as carbon source in enzyme or enzyme mixture production processes.

The process described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure, including definitions will be controlling.

The process may also have additional steps wherein the enzymes harvested from the process are further used to hydrolyzes first ligno-cellulosic biomass. Preferably first ligno-cellulosic biomass and second ligno-cellulosic biomass should be derived from the same grass genus and more preferably derived from the same grass species. It is also preferable that the first ligno-cellulosic biomass upon which the enzymatic hydrolysis to is to be conducted be pre-treated prior to enzymatic hydrolysis.

Also discovered is the enzyme or enzyme mixture made by the process described as well as the ligno-cellulosic biomass which has been hydrolyzed by an enzyme or enzyme mixture produced according to the described process. Thus, this process is applicable growing the host cell in the presence of a first pre-treated ligno-cellulosic biomass and then used to treat the second pre-treated ligno-cellulosic biomass and that the first and second pre-treated ligno-cellulosic biomasses can be derived from the group selected from the same plant genus and the same plant species.

Also claimed in this invention is the enzyme produced according to process described and the ligno-cellulosic biomass which has been hydrolyzed by the enzyme or the processes described.

### Experimental Procedure

The cultivation of the host cell for the production of enzyme(s) proceeds in the following example.

Each host cell cultivation, which in the first instance used Penicillium decumbens as the host cell, started from a spore solution recovered from a PDA-plate seeded with fresh spores seven days before recovery.

1) pre-cultivation which is not part of the claimed cultivation process and

2) host cell cultivation wherein the host cell is grown and the enzyme(s) produced.

### Precultivation:

### Seeding PDA Plate:

1. 500 μl of a previously collected spore solution were dispensed into a PDA plate (3.9% Potato Dextrose Agar medium) prepared as known in the art.

2. 500 μl of sterile 0.9% NaCl solution was dispensed over the spores and the flask gently rotated until the surface was all covered by the liquid.

3. The flask was closed with a cotton plug covered with an aluminium foil and incubated at 30° C. for 7 days.

Spore Solution Recovery:

4. After 7 days, 10 ml of 0.9% NaCl sterile solution was dispensed in the flask.

5. The flask was gently rotated until the liquid became cloudy.

6. As much volume of the NaCl solution was drawn back as possible removing the spore suspension to a sterile tube.

7. The spore solution can be stored indefinitely at 4° C.

### Cultivation Setup Step

Before starting the host cell cultivation it is necessary to set up the cultivation.

Pre-culture medium is prepared as reported below choosing the volume to be at least one-tenth of that of the host cell cultivation phase:

| Pre-Culture Medium Composition | % w/v | g | G |
|---|---|---|---|
| Peptone | 1.00% | 1.00 | 10.00 |
| Pre-treated Ligno-cellulosic | 2.00% | 2.00 | 20.00 |
| biomass | | | |
| KH₂PO₄ | 0.30% | 0.30 | 3.00 |
| MgSO₄ | 0.05% | 0.05 | 0.50 |
| (NH₄)₂SO₄ | 0.20% | 0.20 | 2.00 |
| CaCO₃ | 0.50% | 0.50 | 5.00 |
| Water to add (ml) | | 98 | 980 |
| Glucose | 1.00% | 2.00* | 20.00* |
| Final volume | | 100 | 1000 |

1. The glucose and spore solution are added after sterilization. Spore solution volume is chosen to obtain a final concentration of 5000 CFU/ml.
2. This pre-culture is incubated at 30° C., 170 rpm for 30 h.

### Host Cell Cultivation and Enzyme Production:

The host cell cultivation environment is prepared as reported the table below.

| Host Cell Cultivation Medium composition | |
|---|---|
| | % w/v |
| Pretreated ligno-cellulosic biomass material | 4.50% |
| [on a dry basis] | |
| Urea | 0.50% |
| Tween | 0.10% |
| Glucose (preferably 0.0) | 0.4% |
| | % v/v |
| Pre-Culture Medium volume from previous | 10.0% |
| step | |

1. After sterilization the pH was corrected to 5.3 and the optional simple sugar (glucose) and pre-culture volume were added. The pH is controlled in flasks using different type of buffer solutions (for example 0.1 M phosphate buffer).

2. Host Cell cultivation and enzyme production were carried out at 30° C. setting 170 rpm for flasks and 500 rpm for fermentors. Air supply in fermentors was modified during cell cultivation based upon microorganism requirements.

### Results

The following tables compare activity upon the pre-treated ligno-cellulosic biomass used to feed the host cell. The comparative example (CE1) is the enzyme mixture extraction made via traditional methods where the host cell feed is primarily, if not all glucose. The working example (WE1) was produced as described above wherein the vast majority of the sugars consumed by the host cell were derived from the pretreated ligno-cellulosic biomass. It is clear from the data that when the host cell is cultivated in the presence of ligno-cellulosic biomass, the enzymes produced are far more active to the ligno-cellulosic biomass than enzymes produced from the same host cell strain fed only glucose.

TABLE 1
ENZYME ACTIVITY AFTER 138 h

| | Total cellulasic | β-Glucosidase | Xylanase |
|---|---|---|---|
| | Activity content after 138 h U/ml | | |
| WE1 | 0.68 | 3.31 | 23.86 |
| CE1 | 0.00 | 0.34 | 5.72 |
| | Relative perfromance, % | | |
| WE1 | 100% | 100% | 100% |
| CE1 | 0% | 18% | 8% |

It is apparent from Table 1, that the enzymes derived from the host cell fed the ligno-cellulosic biomass are 5 times more active than those derived from the comparative example.

Table 2 shows the activity development over time of the comparative example. As readily apparent, at no time does the activity of the comparative example exceed the activity of the working example.

TABLE 2
ENZYME ACTIVITY vs TIME

| | Time, hrs | | | | | |
|---|---|---|---|---|---|---|
| | 0 | 65.7 | 89.7 | 113.7 | 137.7 | 161.7 |
| | Total cellulasic Activity, U/ml | | | | | |
| WE1 | 0 | 0.333685 | 0.547819 | 0.733553 | 0.681827 | 0.515875 |
| CE1 | 0 | 0.013113 | 0.014792 | 0 | 0 | 0 |
| | Beta-Glucosidase Activity, U/ml | | | | | |
| WE1 | 0 | 0.528679 | 0.667978 | 2.354583 | 3.307823 | 2.851688 |
| CE1 | 0 | 0.469901 | 0.579264 | 0.46 | 0.341527 | 0.501366 |
| | Xylanase Activity, U/ml | | | | | |
| WE1 | 0 | 20.79375 | 15.4646 | 23.85919 | 16.96183 | 13.13234 |
| CE1 | 0 | 1.607751 | 3.45798 | 5.722076 | 1.339955 | 2.678937 |

Tables 3 and 4 show the results of the process as applied to the various ligno-cellulosic biomasses (corn stover, arundo, populus, wheat straw, miscanthus, and bagasse) as well as arundo with the two other listed host cells. This establishes the method for various types of host cells and ligno-cellulosic biomasses.

TABLE 3
Demonstration of P. Decumbens on Different types of
ligno-cellulosic biomass

| | Time, hrs | | | | |
|---|---|---|---|---|---|
| | 0 | 66 | 90 | 114 | 138 |
| | Fpase Activity, U/ml | | | | |
| Arundo | 0.00 | 0.49 | 0.64 | 0.62 | 0.83 |
| Corn Stover | 0.00 | 0.34 | 0.54 | 0.53 | 0.51 |
| Populus | 0.00 | 0.31 | 0.40 | 0.44 | 0.52 |
| Wheat Straw | 0.00 | 0.26 | 0.53 | 0.47 | 0.63 |
| Miscanthus | 0.00 | 0.47 | 0.65 | 0.78 | 0.97 |
| Bagasse | 0.00 | 0.37 | 0.55 | 0.54 | 0.81 |

TABLE 4
Enzyme Production of Different Microorganisms on Arundo:

| | FPase Activity, U/ml | | | | | |
|---|---|---|---|---|---|---|
| | Time, h | | | | | |
| | 0 | 66 | 90 | 114 | 138 | 168 |
| Penicillium decumbens | 0.00 | 0.49 | 0.64 | 0.62 | 0.83 | |
| Trichoderma reesei | 0.00 | 0.62 | 1.55 | 1.78 | 1.90 | 2.19 |
| RUTC30 | | | | | | |
| Phanerochaete | 0.00 | 0.00 | 0.001 | 0.00215 | 0.006 | 0.31 |
| chrysosporium | | | | | | |
| Trichoderma reesei | 0.00 | 0.05 | 0.41 | 0.63 | 0.75 | 0.85 |
| QM9414 | | | | | | |

FPase is the enzyme activity tested on Filter paper and then the combined with the activity of exo-endo-cellulases and beta-glucosidase, the Ghose assay.

To demonstrate the effect of the added glucose, glucose was added to the cultivation environment in the amount indicated in Table 5 and Table 6. The amount added is the percent by weight of the total cultivation environment. As can be seen in the table, the amount of enzymatic activity is reduced as the amount of glucose increases. The ratio is the ratio of the amount of optional glucose added to the amount of sugars from the pre-treated ligno-cellulosic material.

TABLE 5

| Optional Simple | | | | | | |
|---|---|---|---|---|---|---|
| Sugar (Glucose) | | | | | | |
| added (dry | Ratio of | | | | | |
| weight percent | Simple Sugars | | | | | |
| of cultivation | to sugars | Cultivation Time, hrs | | | | |
| environment) | in biomass | 0 | 66 | 90 | 114 | 138 |

FPase Activity, U/ml

| | | | | | | |
|---|---|---|---|---|---|---|
| 0.0% | 0.00 | 0.000 | 0.441 | 0.532 | 0.576 | 0.688 |
| 0.4% | 0.33 | 0.000 | 0.306 | 0.469 | 0.547 | 0.653 |
| 0.7% | 0.53 | 0.000 | 0.245 | 0.382 | 0.538 | 0.610 |
| 1.4% | 1.12 | 0.000 | 0.170 | 0.314 | 0.557 | 0.587 |
| 2.0% | 1.63 | 0.000 | 0.091 | 0.223 | 0.407 | 0.472 |

TABLE 6

| Optional Simple | | | | | | |
|---|---|---|---|---|---|---|
| Sugar (Glucose) | | | | | | |
| added (dry | Ratio of | | | | | |
| weight percent | Simple Sugars | | | | | |
| of cultivation | to sugars | Cultivation Time, hrs | | | | |
| environment) | in biomass | 0 | 66 | 90 | 114 | 138 |

Xylanase Activity, U/ml

| | | | | | | |
|---|---|---|---|---|---|---|
| 0.0% | 0.00 | 0.00 | 21.68 | 17.22 | 15.41 | 12.83 |
| 0.4% | 0.33 | 0.00 | 18.51 | 16.88 | 14.67 | 10.70 |
| 0.7% | 0.53 | 0.00 | 16.21 | 17.83 | 18.94 | 13.65 |
| 1.4% | 1.12 | 0.00 | 14.85 | 19.40 | 29.54 | 11.11 |
| 2.0% | 1.63 | 0.00 | 10.14 | 12.44 | 13.37 | 12.25 |

The Fpase (filter paper) and Xylanase activity were determined using industry known methods of determining enzymatic activity. The difference being that filter paper was the substrate for Fpase and the xylan mixture described below used as the Xylan substrate.

| | | | |
|---|---|---|---|
| | | Activity | |
| Code | Substrate | type | Preparation |

Main activities

| | | | |
|---|---|---|---|
| FiP | Filter paper | Cellulose | 15 mg Whatman No. 1 |
| | | degrading | filter paper strip |
| | | activity | (0.55 × 3.29 cm). |
| Xyl | Xylan | Xylanase | 2 g Birchwood xylan |
| | | | (Sigma X0502) |
| | | | 70 ml Ultrapure water |
| | | | Heat to boiling with |
| | | | stirring. |
| | | | Cool to room |
| | | | temperature and add 5 ml |
| | | | of 1N buffer |
| | | | stock solution |

We claim:

1-17. (canceled)

18. A process which produces at least one enzyme from a host cell for the hydrolysis of a first pre-treated ligno-cellulosic biomass wherein said process comprises the step of cultivating the host cell which is capable of producing the at least one enzyme for a cultivation time, wherein the cultivation of the host cell occurs in a cultivation environment comprising
the host cell which is Penicillium decumbens,
a second pre-treated ligno-cellulosic biomass comprising complex sugars; the cultivation is done under simple sugar starved conditions of having an amount of optionally added simple sugar or sugars in the range of 0 to 10% by weight of the cultivation environment on a dry basis for a portion of the cultivation time which is at least 50% of the cultivation time;
the cultivation environment is void of added vitamins, minerals, mineral salts, lactose and isopropyl 0-D-1-thiogalactopyranoside; and
the dry matter content by weight of the second pre-treated ligno-cellulosic biomass in the cultivation environment is higher than 4%.

19. The process of claim 18, wherein the dry matter content by weight of the second pre-treated ligno-cellulosic biomass in the cultivation environment is higher than a value selected from the group consisting of 6%, 8%, 10%, 15%.

20. The process of claim 18, wherein the portion of the cultivation time under simple sugar starved conditions is at least 75% of the cultivation time.

21. The process of claim 20, wherein the portion of the cultivation time under simple sugar starved conditions is at least 85% of the cultivation time.

22. The process of claim 21, wherein the portion of the cultivation time under simple sugar starved conditions is at least 90% of the cultivation time.

23. The process of claim 22, wherein the portion of the cultivation time under simple sugar starved conditions is at least 95% of the cultivation time.

24. The process of claim 23, wherein the portion of the cultivation time under simple sugar starved conditions is at least 98% of the cultivation time.

25. The process of claim 18, wherein the portion of the cultivation time under simple sugar starved conditions is the same as the cultivation time.

26. The process of claim 19, wherein the portion of the cultivation time under simple sugar starved conditions is the same as the cultivation time.

27. The process according to claim 18, wherein the amount of optionally added simple sugar or sugars is in the range of 0 to 5% by weight of the cultivation environment on a dry basis.

28. The process according to claim 18, wherein the amount of optionally added simple sugar or sugars is in the range of 0 to 2.5% by weight of the cultivation environment on a dry basis.

29. The process according to claim 18, wherein the amount of optionally added simple sugar or sugars is in the range of 0 to 2.0% by weight of the cultivation environment on a dry basis.

30. The process according to claim 18, wherein the amount of optionally added simple sugar or sugars is in the range of 0 to 1.0% by weight of the cultivation environment on a dry basis.

31. The process according to claim 18, wherein there is no optional added simple sugar or sugars in the cultivation environment.

32. The process of claim 18, wherein the enzyme is harvested by removing the enzyme from the cultivation environment.

33. The process of claim 18, wherein the enzyme is further used to hydrolyze the first ligno-cellulosic biomass and the first ligno-cellulosic biomass and the second pretreated ligno-cellulosic biomass both comprise ligno-cellulosic biomass derived from the same grass genus.

34. The process of claim 18, wherein the enzyme is further used to hydrolyze the first ligno-cellulosic biomass and the first ligno-cellulosic biomass and the second pretreated ligno-cellulosic biomass are derived from the same grass species.

35. The process of claim 33, wherein the first ligno-cellulosic biomass is pretreated prior to enzymatic hydrolysis.

36. The process of claim 34, wherein the first ligno-cellulosic biomass is pretreated prior to enzymatic hydrolysis.
